(19) United States
(12) Patent Application Publication
(10) Pub. No.: US 2023/0143889 A1
(43) Pub. Date: May 11, 2023

(54) WRIST SUPPORT BOARD

(71) Applicants: Marc A. McNeese, St. Augustine, FL (US); Shandra Y. McNeese, St. Augustine, FL (US)

(72) Inventors: Marc A. McNeese, St. Augustine, FL (US); Shandra Y. McNeese, St. Augustine, FL (US)

(21) Appl. No.: 18/148,987

(22) Filed: Dec. 30, 2022

Related U.S. Application Data
(63) Continuation-in-part of application No. 16/873,234, filed on Feb. 28, 2020, now abandoned.
(60) Provisional application No. 62/811,880, filed on Feb. 28, 2019.

(51) Int. Cl. A61M 5/52

(52) U.S. Cl. CPC A61M 5/52

(57) ABSTRACT

A wrist support board has a board with a ventral surface and an opposite dorsal surface, a smooth curve lengthwise, a proximal end to the board and an opposite distal end, a proximal strap, a distal strap, and a center strap. The proximal strap and the distal strap extend from the ventral surface and wrap a patient's arm placed upon the dorsal surface. The center strap extends through slots in the board proximate the vertex of the curve. The center strap enwraps a patient's arm slightly proximal from the patient's wrist. The proximal strap, the distal strap, and the center strap are perpendicular to the board. The three straps secure near their own centers to the board and present two free ends that wrap around a patient's arm. The wrist support board assists in the immobilizing, stabilizing, and hyper-exposure of the trans-radial artery of a patient.

### CROSS-REFERENCE TO RELEATED APPLICATIONS

This non provisional application claims priority to non-provisional application Ser. No. 16/873,234 filed on Feb. 28, 2020 which claims priority to provisional application Ser. No. 62/811,880 filed on Feb. 28, 2019 which are owned by the same inventors.

### BACKGROUND OF THE INVENTION

The wrist support board generally relates to stabilizing a wrist of a patient and more particularly relates to limiting uncontrolled immobilization and exposure of the wrist in relationship to the radial artery.

People suffer various injuries and chronic conditions to their bodies. A person who presents himself for treatment becomes a patient with that injury or chronic condition. Select injuries and conditions call for access to the wrist of a patient for treatment. Alternatively, other injuries and conditions have their access through ankle of a patient for their treatment. The injuries and conditions include carpal tunnel syndrome, aortic defects, other cardiologic conditions and detriments, nerve sheath ruptures, other neurologic deficits and conditions, vascular surgical procedures, orthopedic treatments of joint structures, and other treatments that often involve a catheter. A catheter generally has a form of an elongated flexible tube capable of insertion into a space, a volume, or a tubular structure of a patient.

A common instance of catheter insertion occurs during cardiology. During cardiology, medical staff seek an access to a radial artery of a patient. As a reminder, the radial artery is a major artery in the human forearm. It has a position close to the surface of the underside of the forearm. In a person, when the palm of the hand points upwards, the radial artery also does so. In some situations, medical staff will perform access to the radial artery using a ventral approach and in other situations using a dorsal approach. Medical staff select the approach and then various devices assist in making the approach into the radial artery of the patient. Once medical staff opens access to the radial artery, the medical staff inserts a catheter for additional treatment.

The medical profession has a current problem of supporting a wrist and arm or an ankle and foot in the proper orientation for an access procedure and follow on treatment.

### DESCRIPTION OF THE PRIOR ART

Seeking to support wrists and ankles, the medical staffs across the nation utilize existing technology. To some extent, that technology has limited complexity, almost embarrassing in its simplicity. Medical staffs presently utilize towels, rolled towels, and tape that props up the wrists, arms, ankles, and legs in the proper orientation. Medical staff applies a process of taping the extremity to the procedure table for other medical staff to perform the access procedure. The towels and rolled towels provide a comfortable fit between the extremity and the frame or edge of an operating table. The tape gently but firmly secures the towels and rolled towels to the operating table and the extremity to the rolled towels and towels. Patients who have had prior injuries or played a sport remember the usage of tape and towels from earlier years. However, towels may serve as an infection vector and rolled towels may compress when wetted. The compressed towels may disrupt the proper orientation of an extremity.

The present invention overcomes the disadvantages of the prior art and provides a wrist support board that eliminates the use of tape, towel rolls, and the process of taping an extremity to the operating table. The present invention provides a more ergonomic, comfortable, and safe means to secure and immobilize the trans-radial area of a wrist after arterial access. The wrist support board will see utilization in the securing of vascular sheathes after establishing access, thus preventing accidental dislodgement and massive arterial bleeding. Furthermore, the wrist support board makes it possible for utilization in promoting vascular compression post intervention.

### SUMMARY OF THE INVENTION

Generally, the present invention provides a wrist support board. Though the word patient is used in this specification that also includes veterinary patients and though the phrase medical staff is used that also includes veterinary staff, first responders, and other users of catheters and IV lines. The present invention has a board with a ventral surface and an opposite dorsal surface, a smooth curve lengthwise through the board, a proximal end to the board and an opposite distal end, a proximal strap, a distal strap, and a center strap. The proximal strap and the distal strap extend from the ventral surface and enwrap a patient's arm and fingers placed upon the dorsal surface of the board. The center strap extends through slots in the board proximate the vertex of the curve. The center strap enwraps a patient's arm slightly proximal from the patient's wrist and has a buckler for compression of the patient's arm or wrist. The proximal strap, the distal strap, and the center strap have an orientation perpendicular to the board. The three straps secure proximate their middles to the board and present two free ends for securement following wrapping of the straps around a patient's arm. The invention also has elongated slots to position the center strap for usage in an approach to the pollis artery. The wrist support board assists in the immobilizing, stabilizing, and hyper-exposure of the trans-radial artery of a patient.

There has thus been outlined, rather broadly, the more important features of the invention in order that the detailed description thereof that follows may be better understood and that the present contribution to the art may be better appreciated. The present invention also includes a buckle upon the center strap, the buckle constricts the ends of the center strap together, free ends of the proximal strap and the distal strap secured using hook and loop fastener, a radius of curvature of the board compatible with the wrist of a patient, and hypoallergenic construction.

As stated above, the current problem within the industry involves securing a wrist at an extended angle, alternatively a foot and an ankle, which requires the creative use of towels, rolled towels, and tape. The tape secures the wrist to a towel, the towel within rolled towels, and the rolled towels to an operating table. The present invention solves that problem with a curved board including straps for securement of an arm so that the wrist attains the desired curve. The present invention ensures proper positioning of a wrist, hand, ankle, or foot for select catheter related surgical procedures without the challenges and difficulties associated with tape and rolled towels during preparation of a patient for care and in addition to pre-hospital settings as performed by the EMS or other first responder. Additional features of the invention will be described hereinafter, and which will form the subject matter of the claims attached.

Numerous objects, features and advantages of the present invention will be readily apparent to those of ordinary skill in the art upon a reading of the following detailed description of the presently preferred, but nonetheless illustrative, embodiment of the present invention when taken in conjunction with the accompanying drawings. Before explaining the current embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, the phraseology and terminology employed herein are for the purpose of description and should not be regarded as limiting.

One object of the present invention is to provide a wrist support board that secures and stabilizes a patient's wrist for arterial line monitoring.

Another object is to provide such a wrist support board that prepares a wrist for arterial monitoring during treatment of critically ill or injured patients.

Another object is to provide such a wrist support board that treats minor sprains and strains of the wrist that require hyper-extension immobilization temporarily.

Another object is to provide such a wrist support board that lends simplicity to the use of the invention.

Another object is to provide such a wrist support board that utilizes a one step process to eliminate the multiple steps of current practice.

Another object is to provide such a wrist support board that eliminates the tying or taping of the patient's extremity to an exam table.

Another object is to provide such a wrist support board that has usage in many different medical specialties.

Another object is to provide such a wrist support board that has usage in cardiology, neurology, vascular surgery, and orthopedics among others.

Another object is to provide such a wrist support board that has usage during the universal securing of vascular sheathes following establishment of access, thus preventing accidental dislodgement and massive arterial bleeding.

Another object is to provide such a wrist support board that promotes vascular compression post intervention.

Another object is to provide such a wrist support board that prompts immediate access.

Another object is to provide such a wrist support board that may be utilized in every aspect of cardiology.

Another object is to provide such a wrist support board that may be utilized in every aspect of IV therapy.

Another object is to provide such a wrist support board that may be utilized in every aspect of carpal tunnel therapy.

Another object is to provide such a wrist support board that receives the wrist of a patient in either palm up or palm down orientations.

Another object is to provide such a wrist support board for use with pediatric to geriatric patients and those in between.

Another object is to provide such a wrist support board that has a low cost of manufacturing so the purchasing physicians, nurses, practices, practice groups, hospitals, medical facilities, veterinary facilities, and organizations can readily buy the invention through stores and supply sources.

These together with other objects of the invention, along with the various features of novelty that characterize the invention, are pointed out with particularity in the claims annexed to and forming a part of this disclosure. For a better understanding of the invention, its operating advantages and the specific objects attained by its uses, reference should be made to the accompanying drawings and descriptive matter in which there is illustrated and described a preferred embodiment of the invention.

### BRIEF DESCRIPTION OF THE DRAWINGS

In referring to the drawings,

FIG. 1 shows a top view of the preferred embodiment of the present invention receiving a left arm of a patient for ventral access;

FIG. 2 shows a top view of the present invention receiving a right arm of a patient for dorsal access;

FIG. 3a illustrates a top view of the preferred embodiment of the present invention;

FIG. 4 describes a bottom view of the present invention;

FIG. 5 shows a side elevation view of the preferred embodiment of the present invention;

FIG. 5a shows a side elevation view of the preferred embodiment of the present invention;

FIG. 6 illustrates a side elevation view of the present invention;

FIG. 7 provides a front view of the preferred embodiment of the present invention;

FIG. 8 shows a rear view of the present invention;

FIG. 8a shows a rear view of an alternate embodiment of the present invention;

FIG. 9 provides a perspective view of the present invention; and

FIG. 10 illustrates an alternate embodiment of the invention.

The same reference numerals refer to the same parts throughout the various figures.

### DESCRIPTION OF THE PREFERRED EMBODIMENT

The present invention overcomes the prior art limitations by providing a wrist support board especially during cardiologic or other insertion situations and sees use by nurses, doctors, clinicians, technicians, and related medical staff who insert catheters and related instruments into patients.

A practitioner, or medical staff, follows a procedure or method of use for the present invention that includes these steps. First, the medical staff places the patient's hand and wrist in an appropriately sized invention that hyper-exposes the radial artery. Second, the medical staff secures the device to the selected arm by the hook and loop straps, both proximal and distal, of the invention as later shown. Third, the medical staff then prepares a sterile field on and around the invention as indicated by procedural technique for accessing the radial artery of the patient. And fourth, the medical staff watches the invention as it remains in position for the duration of the procedure, or as per the discretion of the medical staff.

The present invention can be used and characterized similar to other splints and supports for limbs. The present invention though has a critical difference where it has a radius of curvature along its length to support an arm as opposed to present practices of propping an arm and a wrist using towels and other improvised devices.

In the preferred embodiment of the present invention, a wrist support board appears with reference to the drawings in FIG. 1. FIG. 1 has a top view of the invention with its board 2 beneath a left arm A with its palm P shown upward, with hand in extension. The palm has fingers F extending generally from the length of the invention and a thumb T as shown. More particularly, the invention secures the fingers F beneath a distal strap 3, the forearm of the patient beneath a proximal strap 4, and just inwardly from the wrist within a center strap 5 having a buckle 6. In this view, the arm shows its ventral side V upwardly. The invention supports this position of palm, wrist, and arm for a ventral insertion of a catheter of other instrument.

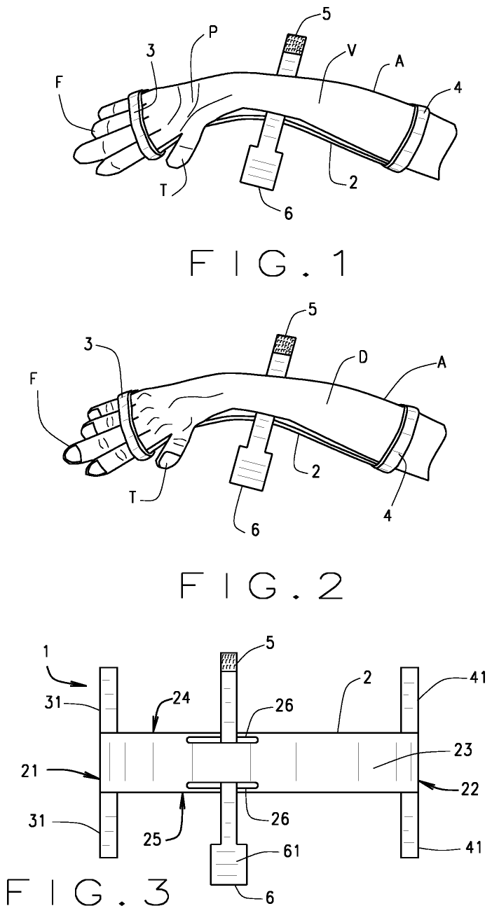

Referring now to FIG. 2, the invention also allows for placement of an arm A with the palm down, that is, knuckles up as shown, with hand in flexion. Here, this figure shows a right hand with its fingers secured beneath the distal strap 3 and with the thumb T extending outwardly from the board 2. The arm has further securement to the board 2 with the center strap 5 and the proximal strap 4. As before, the center strap has its buckle 6. This view has the dorsal surface D of the arm shown towards the reader. The invention supports a hand, a wrist, and an arm placed as shown with knuckles upwardly so medical staff may have a dorsal approach to the radial artery.

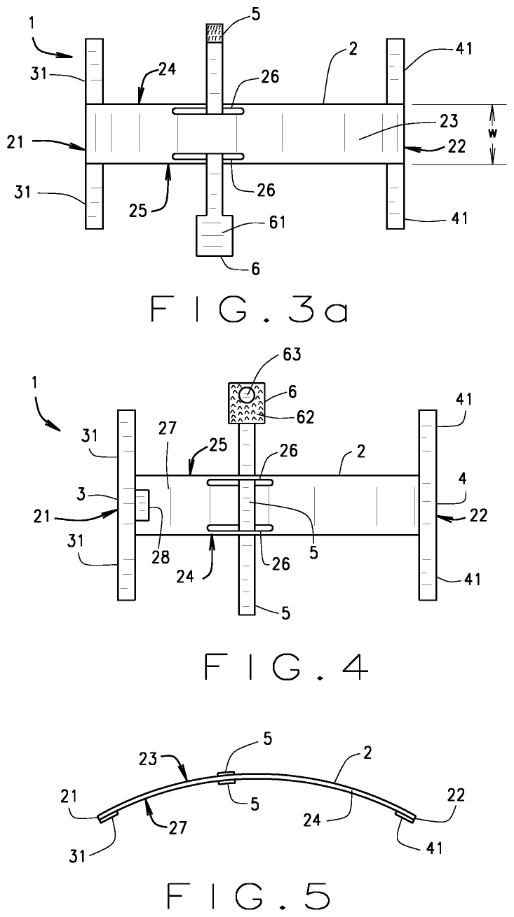

Turning to the invention itself, FIG. 3a shows a top view of the invention before it receives an arm during use shown in FIGS. 1, 2. The invention 1 has an elongated board 2 here shown as rectangular in this view with its dorsal surface 23 visible. This surface abuts an arm during usage. The board has a distal end 21, here shown to the left, and an opposite proximal end 22. The proximal end and the distal end have a generally mutual parallel and spaced apart orientation. Perpendicular to the proximal end, the board 2 has its right edge 24 and its mutually parallel and spaced apart left edge 25. The right edge and the left edge extend for the length of the board and exceed the distal end and the proximal end in length by a factor of at least two. The board has a width, the length of the distal end and of the proximal end proportional to the width of a human arm proximate the wrist. The board has its width here shown as w, to the right. The board also has its elongated, curved shape, of a fixed length measured between the distal end to the proximal end. The board itself is rigid and is thus self-standing. The board also has its inherent thickness shown as constant along its entire length.

Upon the distal end, the distal strap has two free distal portions 31 here shown extending mutually outwardly from the board 2. Opposite those portions, the proximal end has the proximal strap with its two free proximal portions 41. The two free distal portions have cooperating hook and loop fastener sections joined thereto while the two free proximal portions also have cooperating hook and loop fasteners. In the preferred embodiment, the board has two spaced apart slots 26 in mutual registration and positioned slightly proximal of the center of the board. This off center position of the slots permits further positioning of a wrist upon the invention 1. In an alternate embodiment, the slots have a positioned centered upon the length of the board.

The slots 26 have a narrow elongated form positioned inwardly from the right edge and the left edge. The slots have a width sufficient to admit the center strap 5 therein but exclude the buckle 6. That is, the slots in their length exceed the width of the center strap 5. The slots have a width at least twice the width of the center strap. Here, this figure shows slots with a width approximately four times that of the center strap. The center strap has an elongated flexible form. The slots permit movement of the center strap inwardly and outwardly from the centerline of the board so that medical staff may position the buckle 6 at an appropriate place upon a patient's forearm. In a board with two slots, the center strap passes through each slot. In this figure, the buckle 6 has its ventral surface 61 shown as rectangular and this surface also abuts the skin of a patient. The board has its slots 26 generally parallel to the right edge and the left edge but of their length as shown. Here, the slots extend towards the distal end 21 and that allows for positioning the center strap 5 closer to the distal strap 31. In this position, the center strap closes upon the wrist of the patient or up to the carpometacarpal joint. The slots permit usage of the center strap with buckle to secure a patients arm, wrist, or hand for great access capability to the pollis artery during trans radial cardiac catheterization.

In summary, the device 1 has a single component board 2 with a proximal strap 4 and a distal strap 3. Each strap has hook and loop fasteners, often made of Velcro®. The application of the invention upon the patient provides hyper-extension and better exposure of the wrist and radial artery to medical staff for a procedure. Therefore, immobilizing, securing and exposing the access area to a safer and improved ergo-friendly environment.

Next, FIG. 4 shows a bottom view of the invention 1 having its board 2 but with the ventral surface 27 here shown. The ventral surface appears away from the arm upon application of the invention to the arm of a patient. The board has its rectangular form as before and the distal strap 3 upon the distal end 21 with the opposite proximal strap 4 upon the proximal end 22. The distal strap has a thin, flexible, elongated form and the strap secures upon the board's ventral surface in a generally centered position. As in FIG. 3, the distal strap extends outwardly from the centerline of the board, that is, towards the right edge 24 and the left edge 25. The distal strap spans the width of the board as shown. Adjacent to the distal strap, the board 2 has a linking member as at 28. The linking member secures the invention to a supporting surface, such as a table or an armrest, for a patient during a surgical procedure. Preferably, the linking member is a magnetic plate as shown. In alternate embodiments, the linking member may be a magnetic bar, disk, ring, and the like. The magnetic form of the linking member has sufficient field strength to prevent a patient's arm from moving when secured to the invention which is itself secured upon a ferrous portion of the supporting surface, that is, medical furniture. The strength of the magnetic form of the linking member overcomes cushioning, if present, upon the supporting surface. The magnetic form of the linking member has inherent magnetism and does not require electrical current to induce magnetism in the linking member. In alternate embodiments, the linking member may have a planar board construction of polymer, metal, alloy, or other antimicrobial material.

In the preferred embodiment, the center strap 5 has its flexible, thin, elongated form that passes through the two slots 26. The center strap has a free end, here shown proximate the right edge, and the buckle 6 upon the opposite end of the center strap. The buckle has its dorsal surface 62 shown and a knob 63 extending outwardly from the dorsal surface towards the reader. The knob provides fine adjusting and tightening of the center strap into the buckle. One supplier for the knob includes BOA Technology, Inc. of Denver, Colo. And outwardly from the center strap and opposite the distal strap, the invention has its proximal strap 4 with its two free proximal portions 41. The proximal strap also has its form of being thin, flexible, elongated, and the strap secures upon the board's ventral surface, generally centered. As in FIG. 3, the proximal strap extends outwardly from the centerline of the board, that is, towards the left edge and the right edge, or upward and downward in this figure.

FIG. 5 shows a side elevation view of the invention 1 with the right edge 24 towards the reader. The board 2 has its elongated form showing a curve extending along the board's length. The dorsal surface 23 appears upwardly in this figure, that is, the convex appearing surface while the ventral surface 27 appears downwardly akin to a concave surface. The board has its distal end 21 towards the left of the figure and the proximal end 22 towards the right of the figure and slightly higher than the distal end. This apparent elevation difference between the two ends has a relation to the length of curvature before and after the center strap 5. The board has a radius of curvature and a length of curvature proportional and compatible to a wrist of a human arm. The curve of the board has a shorter, or lesser portion, ahead of the center strap 5, that is, between the center strap and the distal end 21 and a longer, or greater portion, behind the center strap 5, that is, between the center strap and the proximal end 22. Typically, the forearm of a patient rests upon the greater portion of the curve while the wrist and hand of a patient rest upon the lesser portion of the curve. As before, the distal end 21 has the distal strap 3 here showing its free distal end 31 on edge and the proximal end 22 has the free proximal end 41 shown on edge from the proximal strap 4. In this view, the center strap has its free portion shown thus obscuring the buckle.

FIG. 5a illustrates a side elevation view of the invention 1 with the right edge 24 in the foreground and the drawing of the invention annotated for its curve geometry. The invention as suggested above and below has a curve to its board compatible with a human forearm, wrist, and hand. The invention has its length, shown as I, denoted below the center of the board in the figure. The board length, l, is at least twice that of its width, w. Moreover, the board has its chord height, c, at the left edge 24 above an imaginary horizontal line between the distal end 21 and the proximal end 22. The chord height reaches its maximum at the center of the length of the invention. The chord height c is at least 75% of the board's width, w, and at least two inches.

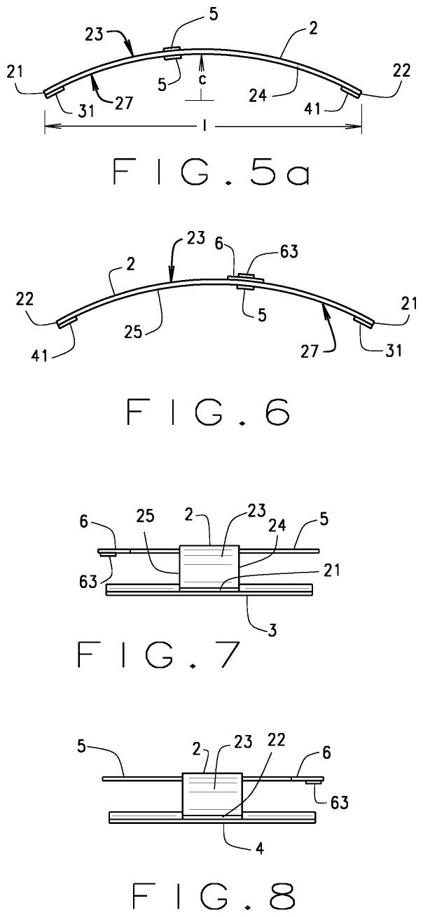

Then FIG. 6 has an opposite side elevation view from that of FIG. 5. As before, the board 2 has its dorsal surface 23 upwardly and its ventral surface 24 downwardly, its proximal end 22 and opposite distal end 21, the proximal strap 4 and the distal strap 3, the free distal end 41, and the free proximal end 31 showing. Between the two straps and near the vertex of the curvature of the board, the board has the center strap with the buckle 6 extending outwardly towards the reader. The buckle has its generally rectangular form with the knob 63 extending further downwardly in this figure, that is, towards the center of curvature for the board. As also suggested in FIG. 5, this figure shows the distal strap, the center strap and buckle, and the proximal strap each adjoining the ventral surface of the board.

Rotating the invention, FIG. 7 provides a front view of the invention with the distal end 21 in the foreground with its distal strap 3 extending outwardly, that is, left and right in this figure. From the distal end rearward, the board 2 curves upwardly and has its left edge 25 and its opposite right edge 24. Proximate the vertex of the board, that is, the top of this figure, the board has the center strap 5 to the right and the buckle 6 with the knob 63 shown downwardly. During usage, the free end of the center strap inserts into the buckle and medical staff secures the knob, so the buckle has an appropriate compression upon the arm of a patient.

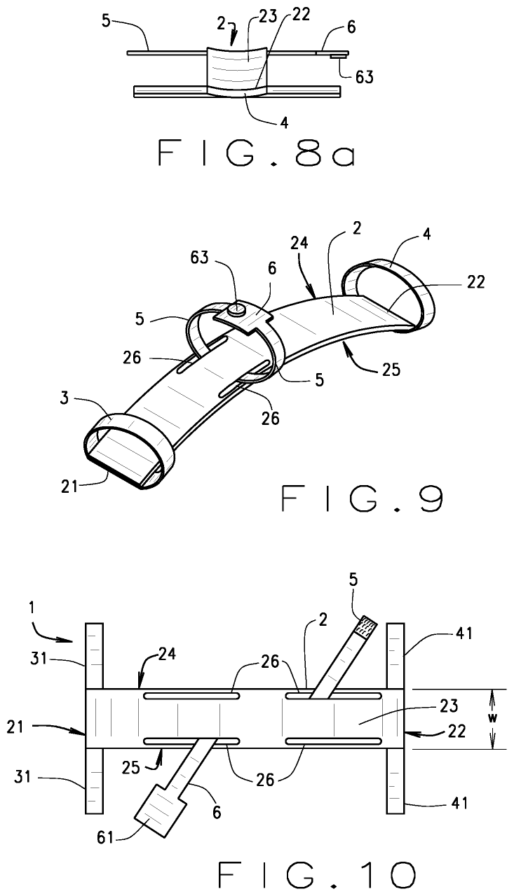

Opposite FIG. 7, FIG. 8 shows a rear view of the invention but with the proximal end 22 in the foreground and its proximal strap 4 extending its free proximal portions 41 outwardly. From the proximal end rearward, the board curves upwardly for an apparent greater height as this portion of the board has a greater length curved than that shown in FIG. 7 previously. As before, the center strap 5 and its counterpart buckle 6 with knob 63 extend outwardly from the vertex of the board. Though FIGS. 7, 8 show the board 2 with a generally flat left to right cross section, the Applicants foresee an alternate curved shape across the width of the board. This curve receives the wrist and arm appropriately as shown in FIG. 8a.

FIG. 8a has another rear view showing the board on end, here showing the proximal end 22, and displaying a concave curvature across the width, w, of the board. The concave curvature across the width accepts the cross sectional shape of a forearm, centers the forearm across the width of the board, and guides the forearm to rest upon the dorsal surface 23 without slipping over the left edge and the right edge.

FIG. 9 shows the invention 1 in a perspective view with its proximal strap 4 having the proximal free ends 41 secured and forming the strap 4 into a loop, with its distal strap 3 also have its distal free ends 31 securing this strap 3 into a loop, and the center strap 5 having its free end inserted into the buckle 5. The free end of the center strap engages the knob so that rotation of the knob in one direction constricts the center strap upon the forearm of a patient. In this view, the board has its left edge 25 towards the reader and its right edge 24 upwardly and away from the reader.

In an alternate embodiment, the invention has dual compression capability. The board 2 supports a patient's arm and wrist in a position where the palm faces up, or supinated position. The board also follows developments in medicine where practitioners have accessed the pollis artery which runs along the pronated position of the thumb and has great access capability for trans radial cardiac catheterization. The board 2 also supports a patient's arm and wrist in a position where the palm faces down, or pronated position. The invention's board allows for usage with supinated and pronated positions of a patient's palm. In usage, a practitioner rotates the board one hundred eighty degrees about an axis perpendicular to its length. In this embodiment, the slots have additional length towards the proximal end so the center strap 5 reaches a patient's wrist positioned on the proximal end and his forearm upon the greater portion of the curve. This alternate embodiment has the slots and curve cooperate, so the slots allow the center strap to migrate towards the pollis artery. This alternate embodiment allows the invention to support two access points for the physician to choose for a catheterization procedure.

And, FIG. 10 shows a top view of an alternate embodiment of the invention before receiving an arm placed upon it in usage. The invention 1 has as before its elongated board 2, with its dorsal surface 23 visible, its distal end 21 and opposite proximal end 22, right edge 24 and left edge 25 with the lengths and orientations as previously shown. The board has its width here shown as w, to the right. The board has two pairs of slots 26, that is, four slots in total, generally parallel to the right edge and the left edge but of greater length than the slots shown in FIG. 3a above. Here, the slots extend further towards the distal end 21 and the proximal end 22 than in the preferred embodiment. This lengthening of the slots allows for positioning the center strap 5 closer to the distal strap 31 if desired by the medical staff user. In this position, the center strap closes upon the wrist of the patient or up to the carpometacarpal joint. The slots of this embodiment permit usage of the center strap with buckle to secure a patients arm, wrist, or hand for great access capability to the pollis artery during trans radial cardiac catheterization.

Unlike in the previous figures, here the center strap 5 occupies diagonally opposite slots as shown with the buckle 6 and its ventral surface 61 shown diagonally opposite the free end of the center strap 5. This number of slots and the length of the slots allows for repositioning of the center strap readily and for use upon a diagonal. More particularly, the four slots—of the two pairs—have elongated and slender form, each pair of slots in this figure appear mutually parallel and spaced apart, and each pair of slots has its position inwardly from the left edge or the right edge. The center strap passes through one of each pair of slots, so it attains one portion of an X like shape. When medical staff utilizes two center straps 5 in a diagonal arrangement, the straps 5 provide further securing of the invention upon a patient's arm against movement linearly upon the arm.

From the aforementioned description, a wrist support board has been described. The wrist support board is uniquely capable of strapping the hand and forearm of a person into an arcuate form for select medical procedures. The wrist support board, and its various components may be manufactured from many materials, including but not limited to, polyester, steel, aluminum, brass, bronze, polymers, high density polyethylene, polypropylene, ferrous and non-ferrous metals, their alloys, and composites. The wrist support board would be made from a stamp molding process and have sizes of small, medium, and large respectively to compensate for all age groups. With the device being a single item with hook and loop, or Velcro®, secured straps, all of the elements of the invention would be considered necessary.

As such, those skilled in the art will appreciate that the conception, upon which this disclosure is based, may readily be utilized as a basis for the designing of other structures, methods and systems for carrying out the several purposes of the present invention. Therefore, the claims include such equivalent constructions insofar as they do not depart from the spirit and the scope of the present invention.

While the present invention has description above of its preferred embodiment, it will be understood that it is not intended to limit the invention to these embodiments. Instead, it is intended to cover all alternatives, modifications and equivalents as may be included within the spirit and scope of the present invention as defined by the appended claims.

We claim:

1. A medical device comprising:
a board having an elongated, curved shape, a distal end, and an opposite proximal end, said board having a fixed length from said distal end to said proximal end and being rigid, said board being self-standing;
a distal strap joining to said distal end, said distal strap extending perpendicular to the length of said board;
a proximal strap opposite said distal strap, said proximal strap joining to said proximal end, said proximal strap extending perpendicular to the length of said board and generally parallel to said distal strap;
a center strap connecting to said board between said distal strap and said proximal strap;
a buckle upon said center strap wherein said buckle constricts said center strap during usage; and
wherein said medical device secures an arm of a patient placed therein for dorsal access and for ventral access.

2. The medical device of claim 1 further comprising:
said board having a right edge and an opposite left edge, said right edge and said left edge having a mutually parallel and spaced apart orientation, said right edge and said left edge each having a greater length than said distal end.

3. The medical device of claim 1 further comprising:
said buckle having a ventral surface and an opposite dorsal surface, the ventral surface of said buckle is adapted to contact a patient's arm, and a knob upon said dorsal surface engaging said center strap when inserted beneath said knob; and
wherein rotation of said knob clockwise constricts said center strap beneath said knob and wherein rotation of said knob counterclockwise releases said center strap.

4. The medical device of claim 2 further comprising:
two slots, each slot locating inwardly from said left edge and said right edge; and
said center strap passing through each of said slots.

5. The medical device of claim 4 further comprising:
said slots having an elongated shape mutually parallel and parallel to said left edge and said right edge respectively.

6. The medical device of claim 2 further comprising:
said curved shape of said board extending for the length of said board;
said curve shape of said board is adapted to fit a wrist of a patient; and
said board having a constant thickness for the length of said board.

7. The medical device of claim 6 further comprising:
said curve of said board having a greater portion between said slots and said distal end and a lesser portion between said slots and said proximal end.

8. The medical device of claim 2 further comprising:
said board having a ventral surface and an opposite dorsal surface; and
a linking member upon said board joining to said ventral surface.

9. The medical device of claim 2 further comprising:
four slots arranged in two pairs, each slot being elongated and slender, each pair of slots being mutually parallel and spaced apart, each pair of slots locating inwardly from one of said left edge and said right edge; and
said center strap passing through one of each of said pairs of slots.

10. A medical device comprising:
a board having an elongated, curved shape, a distal end, and an opposite proximal end, said board having a fixed length from said distal end to said proximal end and being rigid, said board being self-standing;
a distal strap joining to said distal end, said distal strap extending perpendicular to the length of said board;
a proximal strap opposite said distal strap, said proximal strap joining to said proximal end, said proximal strap extending perpendicular to the length of said board and generally parallel to said distal strap;
a center strap connecting to said board between said distal strap and said proximal strap;
a buckle upon said center strap wherein said buckle constricts said center strap during usage, said buckle having a ventral surface and an opposite dorsal surface, the ventral surface of said buckle is adapted to contact a patient's arm, and a knob upon said dorsal surface engaging said center strap when inserted beneath said knob, and rotating said knob clockwise constricting said center strap beneath said knob and rotating said knob counterclockwise releasing said center strap; and
wherein said medical device secures an arm of a patient placed therein for dorsal access and for ventral access.

11. The medical device of claim 10 further comprising:
said board having a right edge and an opposite left edge, said right edge and said left edge having a mutually parallel and spaced apart orientation, said right edge and said left edge each having a greater length than said distal end;
two slots, each slot locating inwardly from said left edge and said right edge, each of said slots having an elongated rectangular shape and being mutually parallel and parallel to said left edge and said right edge respectively; and
said center strap passing through each of said slots.

12. The medical device of claim 10 further comprising:
said curve of said board extending along the length of said board, said curve of said board adapted to fit a wrist of a patient; and
said curve of said board having a greater portion between said slots and said distal end and lesser portion between said slots and said proximal end.

13. The medical device of claim 12 further comprising:
said board having a width and said curve of said board extending upwardly from said distal end and said proximal end for a chordal height proximate the adjoining of said greater portion and said lesser portion;
said chordal height being at least seventy-five percent of the width of said board; and
said board having a constant thickness for the length of said board.

14. The medical device of claim 13 further comprising:
said board being concave across its width for the length of the board; and
said board having a ventral surface and an opposite dorsal surface.

15. The medical device of claim 14 further comprising:
a linking member upon said board joining to said ventral surface;
said linking member having a planar form; and
said linking member being one of polymer, metal, alloy, and composite.

16. The medical device of claim 10 further comprising:
said board having a right edge and an opposite left edge, said right edge and said left edge having a mutually parallel and spaced apart orientation, said right edge and said left edge each having a greater length than said distal end;
four slots arranged in two pairs, each slot being elongated and slender, each pair of slots being mutually parallel and spaced apart, each pair of slots locating inwardly from one of said left edge and said right edge;
said center strap passing through one of each of said pairs of slots;
said curve of said board extending along the length of said board, said curve of said board adapted to fit a wrist of a patient;
said curve of said board having a greater portion between said slots and said distal end and lesser portion between said slots and said proximal end.
said board having a width and said curve of said board extending upwardly from said distal end and said proximal end for a chordal height proximate the adjoining of said greater portion and said lesser portion;
said chordal height being at least seventy-five percent of the width of said board;
said board having a constant thickness for the length of said board;
said board being concave across its width for the length of the board;
said board having a ventral surface and an opposite dorsal surface; and
a linking member upon said board joining to said ventral surface.

17. A medical device comprising:
a center strap;
a buckle upon said center strap wherein said buckle constricts said center strap during usage, said buckle having a ventral surface and an opposite dorsal surface, the ventral surface of said buckle is adapted to contact a patient's arm, and a knob upon said dorsal surface engaging said center strap when inserted beneath said knob, and rotating said knob clockwise constricting said center strap beneath said knob and rotating said knob counterclockwise releasing said center strap;
a distal strap and a spaced apart proximal strap, said center strap locating between said distal strap and said proximal strap;
a board having an elongated, curved shape, a distal end, and an opposite proximal end, said board having a fixed length from said distal end to said proximal end and being rigid, said board being self-standing;
said distal strap extending perpendicular to the length of said board, said proximal strap joining to said proximal end, said proximal strap extending perpendicular to the length of said board and generally parallel to said distal strap;
said board having a right edge and an opposite left edge, said right edge and said left edge having a mutually parallel and spaced apart orientation, said right edge and said left edge each having a greater length than said distal end,
said board having a width and said curve of said board extending upwardly from said distal end and said proximal end for a chordal height being at least seventy five percent of the width of said board;
at least two slots, each slot locating inwardly from said left edge and said right edge, each of said at least two slots having an elongated rectangular shape and being mutually parallel and parallel to said left edge and said right edge respectively;
said a center strap connecting to said board through said at least two slots and connecting to said board between said distal strap and said proximal strap;
said curve of said board extending along the length of said board, said curve of said board adapted to fit a wrist of a patient;
said curve of said board having a greater portion between said at least two slots and said distal end and a lesser portion between said slots and said proximal end, said chordal height locating proximate where said greater portion adjoins said lesser portion; and
wherein said medical device secures an arm of a patient placed therein for dorsal access and for ventral access.

18. The medical device of claim 17 further comprising:
said at least two slots including four slots arranged in two pairs, each slot being elongated and slender, each pair of slots being mutually parallel and spaced apart, each pair of slots locating inwardly from one of said left edge and said right edge;
said center strap passing through one of each of said pairs of slots;
said board being concave across its width for the length of the board; and
said board having a ventral surface and an opposite dorsal surface.
